(19) United States
(12) Patent Application Publication
(10) Pub. No.: US 2022/0175272 A1
(43) Pub. Date: Jun. 9, 2022

(54) BIOLOGICAL SAMPLE CAPTURING DEVICE

(71) Applicant: AGSCENT PTY LTD, Carwoola (AU)

(72) Inventor: Bronwyn DARLINGTON, Carwoola (AU)

(21) Appl. No.: 17/600,388

(22) PCT Filed: Mar. 31, 2020
(86) PCT No.: PCT/AU2020/050318

§ 371 Date: Sep. 30, 2021

(30) Foreign Application Priority Data
Mar. 31, 2019 (AU) 2019901085

(51) Int. Cl. A61B 5/097; A61B 5/08

(52) U.S. Cl. CPC A61B 5/097; A61B 2560/0406; A61B 2503/40; A61B 5/082

(57) ABSTRACT

In one embodiment, the present invention provides a biological sample capture device comprising a body including a chamber, the chamber being arranged to sealingly hold a biological sample, a first valve in communication with the chamber, wherein the first valve allows a biological sample to enter the chamber, a second valve in communication with the chamber, wherein the second valve is arranged to expel air from the chamber upon a biological sample entering the chamber via the first valve.

### TECHNICAL FIELD

The present technology relates to a biological sample capturing device. In one embodiment, the invention relates to a device for capturing a breath sample from an animal, such as a cow, pig or sheep.

### BACKGROUND

It is known that biological samples from animals can yield a vast amount of information about the animal. For example, the blood of an animal carries certain chemical compounds which can be separated from a blood sample collected from the animal and analysed to determine the presence or absence of any one of a number of medical conditions, from disease and deficiency, through to pregnancy.

It is also known that some of these compounds are capable of crossing the lung barrier and traces of the compounds may be present in the exhaled breath of an animal. The presence and/or amount of the compound present in the analysis of a chemical sample of an animal's breath can be used to determine the presence or absence of any one of a number of medical conditions. For example, “breathalysers” are well known examples of electronic devices that include a sensor which, when presented with a sample of a person's breath, is capable of detecting the blood alcohol level of the person. One of the advantages of using breath as a biological sample is that the collection of a breath sample is generally less invasive and easier to collect.

It is with at least some of these issues in mind that the present invention has been developed.

In the context of the specification, the term “breath” refers to a mixture of gas and liquid secreted/diffused into the lungs of an animal from the blood of the animal, and subsequently exhaled from the lungs of the animal via the nose and/or mouth of the animal and consequently “breath sample” refers to a sample of the mixture of gas and liquid, including Volatile Organic Compounds (VOCs).

### SUMMARY OF THE TECHNOLOGY

In a first aspect, there is provided a biological sample capture device comprising a body including a chamber, the chamber being arranged to seal within it a biological sample, a first valve in communication with the chamber, wherein the first valve allows a biological sample to enter the chamber, a second valve in communication with the chamber, wherein the second valve is arranged to expel air from the chamber upon a biological sample entering the chamber via the first valve.

Broadly, in one aspect of the present invention there is also provided a mask portion arranged to seal-fit over the first valve, the mask portion being arranged to fit over a nostril of an animal. In one embodiment, the mask is shaped to provide a sealing fit over a single nostril of the animal, such that the exhaled breath of the animal is captured and directed towards the first valve, causing the first valve to open and the exhaled breath of the animal to enter the chamber. Correspondingly, the temporary increase in pressure in the chamber caused by the exhaled breath entering the chamber causes the second valve to open to allow air trapped inside the chamber to escape until the pressure within the chamber is equalised with the pressure outside the chamber. Upon the animal finishing their exhalation, the pressure caused by the exhalation ceases and the one-way valves close, thereby sealing the sample.

In one embodiment, there is also provided a first cap arranged to, upon removal of the mask portion, seal-fit over the first valve, to provide a secondary seal. The first cap may also include a permeable portion composed of a self-sealing material such as a silicone rubber, arranged to allow a needle to penetrate the cap, to thereby draw out a sample of the biological substance, such as the breath of an animal.

Correspondingly, there may also be provided a second cap arranged to seal-fit over the second valve, to provide another secondary seal.

It will be understood that the first and second caps may be removably attached to the device utilising any suitable means, including a screw fit, a twist fit, a pressure fit, a snap fit, or any other form of fitting that provides an adequate seal.

In one embodiment, where the chamber is substantially cylindrical in shape, with the first and second valves being disposed at each end of the substantially cylindrical chamber, there is further provided a threaded portion at each end of the body, arranged to receive a complementary threaded portion disposed on one of mask portion, the first cap or the second cap.

Broadly in one aspect of the present technology there is provided a series of joints, the joints being disposed at each end of the body, wherein the joints and the chamber comprise the body. In one embodiment, the joints and the substantially cylindrical chamber are integrally formed as a single piece. In another embodiment, the chamber is formed of a glass, metal or other material resistant to corrosion and non-permeable.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is a side view of a biological sample capture device in accordance with an embodiment of the invention;

FIG. 2 is a perspective view of a biological sample capture device with a mask portion in accordance with an embodiment of the invention;

FIG. 3 is a perspective view of a biological sample capture device with caps in accordance with an embodiment of the invention;

FIG. 4 is an exploded view of a biological sample capture device with a mask portion in accordance with an embodiment of the invention;

FIG. 5 is an exploded view of a biological sample capture device with caps in accordance with an embodiment of the invention; and

FIGS. 6a and 6b show a device in accordance with an embodiment of the invention in use.

### DETAILED DESCRIPTION OF EMBODIMENTS

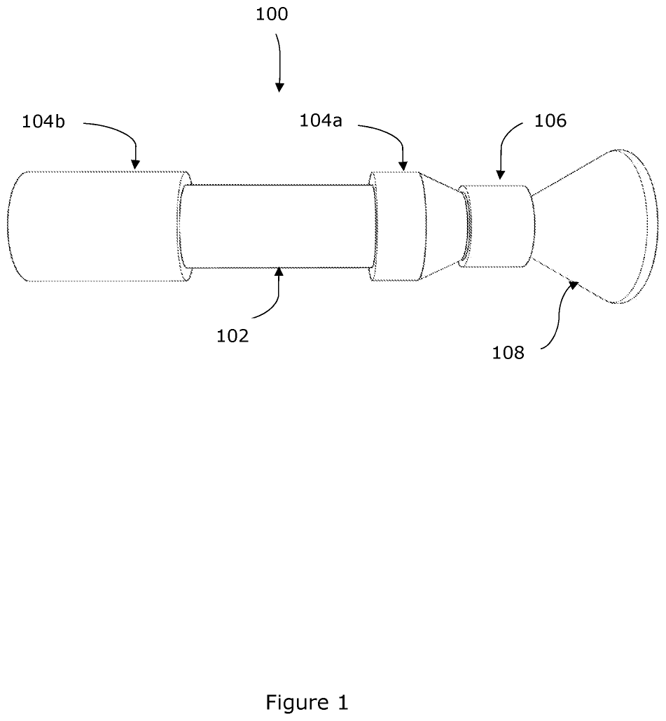

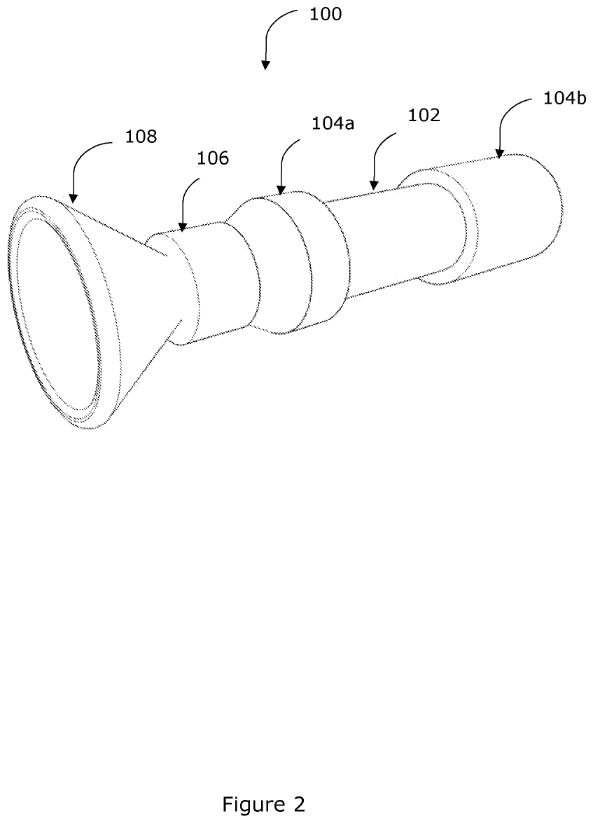

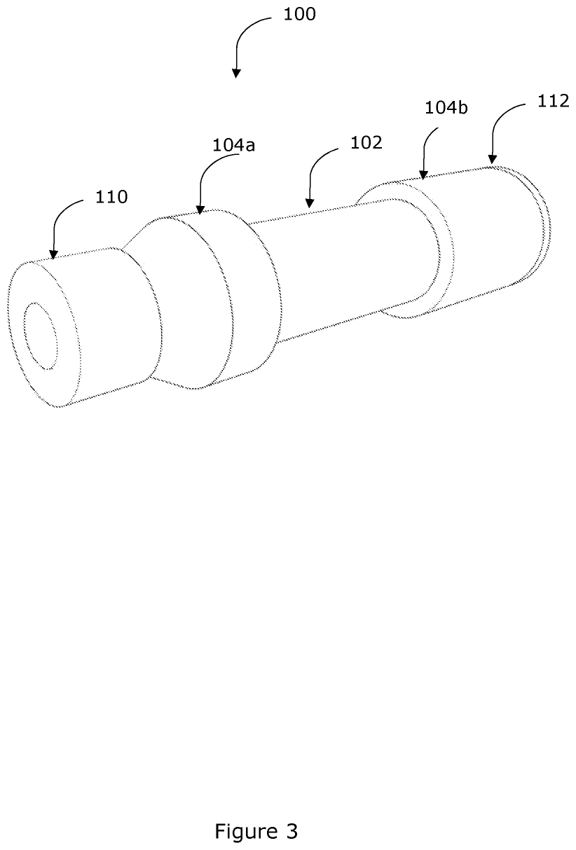

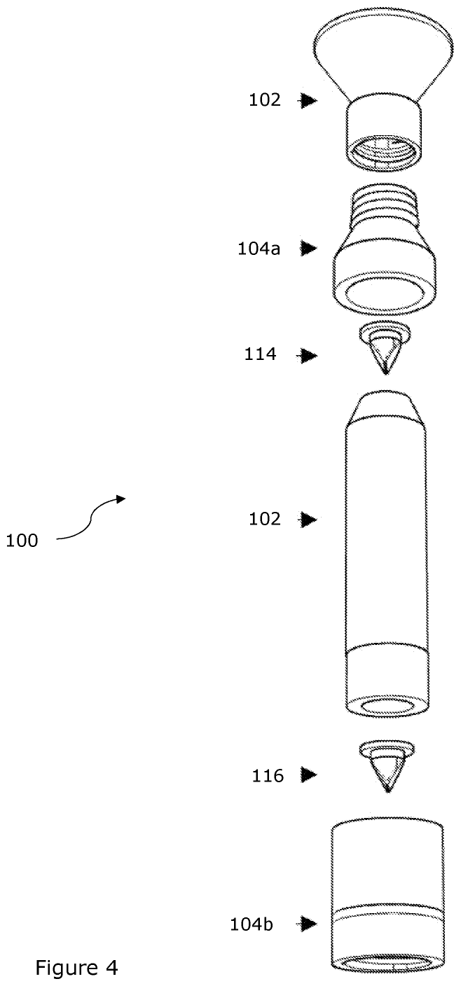

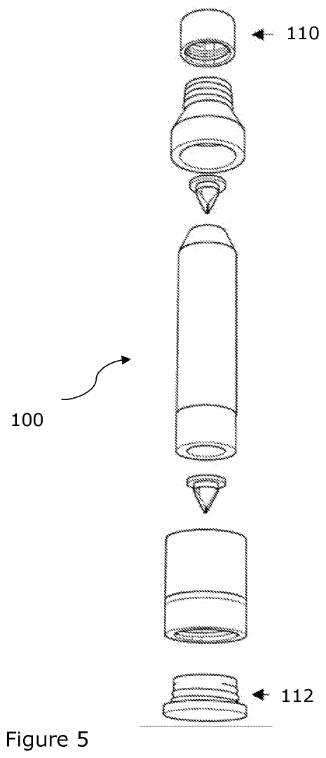

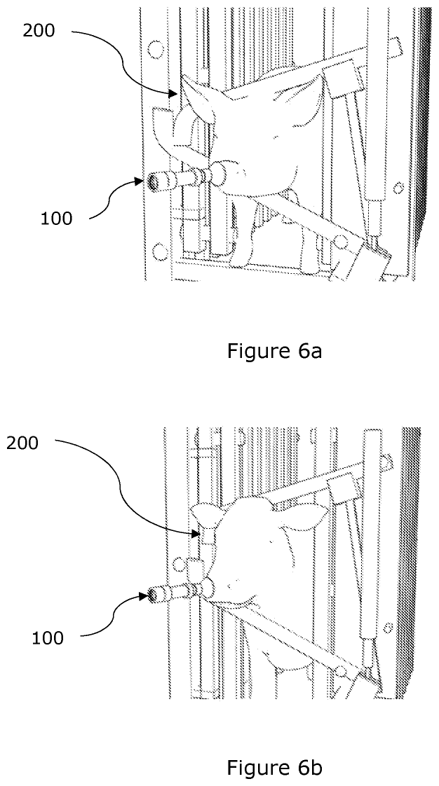

In the following detailed description of FIGS. 1 to 6b, like numerals across different Figures refer to like features and/or integers.

Referring to FIGS. 1 through 6b, there are shown various view of a device capable of capturing biological samples. In one embodiment, the device is a breath capturing device 100 capable of capturing the breath of an animal 200, for later analysis.

An embodiment of the invention is provided in FIG. 1, wherein the 100 is shown. The device 100 includes a body 102 that includes a chamber, which in the embodiment is shown as a generally cylindrical canister-like object that is made of a hard material that is resistant to contamination and/or easy to clean, such as glass, stainless steel, or any other corrosion resistant material. The body is arranged to capture and hold a sample of the breath of an animal.

The biological sample capture device 100 has a body including a chamber, the chamber being arranged to sealingly hold a biological sample of breath, a first valve 114 in communication with the chamber, wherein the first valve 114 allows a biological sample to enter the chamber, a second valve 116 in communication with the chamber, wherein the second valve 116 is arranged to expel air from the chamber upon a biological sample entering the chamber via the first valve. It will be understood that while the embodiment shown in the Figures has the first valve 114 and the second valve 116 disposed at opposite ends of the device 100, the valves may be disposed at any location on the device 100.

Broadly, in one aspect of the present invention there is also provided a mask portion 106 arranged to sealingly fit over the first valve 114, the mask portion 106 being arranged to fit over the nostril of an animal using portion 108. In one embodiment, the mask 106 is shaped to provide a sealing fit over a single nostril of the animal, such that the exhaled breath of the animal is captured and directed towards the first valve, causing the first valve to open and the exhaled breath of the animal to enter the chamber. Correspondingly, the temporary increase in pressure in the chamber caused by the exhaled breath of the animal entering the chamber causes the second valve to open to allow air trapped inside the chamber to escape until the pressure is equalised in the chamber. Upon the animal finishing their exhalation, the pressure caused by the exhalation ceases and the one-way valve closes, thereby sealing the sample.

In one embodiment, there is also provided a first cap 110 arranged to, upon removal of the mask portion, sealingly fit over the first valve, to provide a secondary seal. The first cap 110 may also include a permeable portion (not explicitly shown in the figures) composed of a self-sealing material such as a silicone rubber, arranged to allow a needle to penetrate the cap, to thereby draw out a sample of the biological substance, such as the breath of an animal.

Correspondingly, there may also be provided a second cap 112 arranged to sealingly fit over the second valve 116, to provide another secondary seal.

In one embodiment, where the chamber is substantially cylindrical in shape, with the first valve 114 and second valve 116 being disposed at each end of the substantially cylindrical chamber, there is further provided a threaded portion at each end of the body, arranged to receive a complementary threaded portion disposed on one of mask portion, the first cap or the second cap. It will be understood that the first and second caps may be removably attached to the device utilising any suitable means, including a screw fit, a twist fit, a pressure fit, a snap fit or any other form of fitting that provides an adequate seal.

Broadly in one aspect of the present technology there is provided a series of rubberised joints 104a and 104b, the joints being disposed at each end of the body, wherein the joints 104a and 104b and the chamber comprise the body. In one embodiment, the joints and the substantially cylindrical chamber are integrally formed as a single piece. In another embodiment, the chamber is formed of a glass, metal or other material resistant to corrosion.

### Advantages

One of the advantages of the embodiments and broader invention described herein is that the device provides a cost effective, reliable and reusable device for capturing a breath sample. This is particularly important in dusty, wet and otherwise “dirty” environments, where the collection of an animal's breath is difficult.

Moreover, the shape of the device, including the mask portion, and the relative size of the device, allows a user to easily collect a breath sample from an animal without causing stress or distress to the animal. The mask portion may be made in different sizes for different animals, and is shaped such that, for some animals, such as cattle, the mask portion fits over one nostril. Therefore, a sample can be captured by simply pressing the mask portion to the nostril of a cow (for example), and the cow's normal exhalation of breath causes the sample to travel into the chamber, with the first and second valves opening due to the exhalation pressure and causing the sample to flow into the chamber automatically.

It is noted that the chamber in question is arranged to capture Volatile Organic Compounds (VOCs) present in the exhalation of all complex animals for later testing at a laboratory. However, it will be understood that the device described and defined herein may also be adapted to integrate or mate with a testing device, such as chemical testing device or an electronic testing device, such that the capture of breath and the analysis of the captured breath occurs contemporaneously. Such variations and permutations are within the purview of a person skilled in the art.

### Definition and Clarification of Terms and Intent

Throughout this specification, unless the context requires otherwise, the word “comprise” or variations such as “comprises” or “comprising”, will be understood to imply the inclusion of a stated integer or group of integers but not the explicit exclusion of any other integer or group of integers.

Those skilled in the art will appreciate that the embodiments described herein are susceptible to obvious variations and modifications other than those specifically described and it is intended that the broadest claims cover all such variations and modifications. Those skilled in the art will also understand that the inventive concept that underpins the broadest claims may include any number of the steps, features, and concepts referred to or indicated in the specification, either individually or collectively, and any and all combinations of any two or more of the steps or features may constitute an invention.

Where definitions for selected terms used herein are found within the detailed description of the invention, it is intended that such definitions apply to the claimed invention. However, if not explicitly defined, all scientific and technical terms used herein have the same meaning as commonly understood to one of ordinary skill in the art to which the invention belongs.

1. A hand-held biological sample capture device comprising a body including a chamber, the chamber being arranged to sealingly hold a biological sample, a pressure actuated first one-way valve in communication with the chamber, wherein, upon receiving a biological sample in the form of exhaled breath of an animal, the first one-way valve allows the biological sample to enter the chamber, and a pressure actuated second one-way valve in communication with the chamber, wherein the second one-way valve is arranged to expel air from the chamber upon a biological sample entering the chamber via the first one-way valve.

2. A device in accordance with claim 1, further comprising a mask portion arranged to sealingly fit over the first valve, the mask portion being arranged to fit over the nostrils of an animal.

3. A device in accordance with claim 2, wherein the mask is shaped to provide a sealing fit over a single nostril of the animal, such that the exhaled breath of the animal is captured and directed towards the first valve.

4. A device in accordance with claim 3, wherein the mask, in use, causes the exhaled breath of the animal to be directed towards the first valve causing the first valve to open and the exhaled breath of the animal to enter the chamber.

5. A device in accordance with claim 4, wherein the temporary increase in pressure in the chamber caused by the exhaled breath entering the chamber causes the second valve to open to allow air trapped inside the chamber to escape until the pressure is equalised in the chamber.

6. A device in accordance with claim 5, wherein upon the animal finishing their exhalation, the pressure caused by the exhalation ceases and the one-way valve closes, thereby sealing the sample.

7. A device in accordance with claim 1, further including a first cap arranged to, upon removal of the mask portion, sealingly fit over the first valve, to provide a secondary seal.

8. A device in accordance with claim 7, wherein the first cap includes a permeable portion composed of a self sealing material such as a silicon rubber, arranged to allow a needle to penetrate the cap, to thereby draw out a sample of the biological substance, such as the breath of an animal.

9. A device in accordance with claim 1, further including a second cap arranged to sealingly fit over the second valve, to provide another secondary seal.

10. A device in accordance with claim 1, wherein the chamber is substantially cylindrical in shape.

11. A device in accordance with claim 1, wherein the first and second valves are disposed at each end of the substantially cylindrical chamber.

12. A device in accordance with claim 1, wherein there is further provided a threaded portion at each end of the body, arranged to receive a complementary threaded portion disposed on one of mask portion, the first cap or the second cap.

13. A device in accordance with claim 1, further including a series of joints, the joints being disposed at each end of the body, wherein the joints and the chamber comprise the body.

14. A device in accordance with claim 13, wherein the joints and the substantially cylindrical chamber are integrally formed as a single piece.

15. A device in accordance with claim 1, wherein the chamber is formed of a glass, metal or other material resistant to corrosion.
